(19) United States
(12) Patent Application Publication
(10) Pub. No.: US 2014/0277111 A1
(43) Pub. Date: Sep. 18, 2014

(54) OVAL VASCULAR CLOSURE DEVICE AND METHODS

(71) Applicant: ST. JUDE MEDICAL PUERTO RICO LLC, Caguas, PR (US)

(72) Inventor: Zachary J. Tegels, Minneapolis, MN (US)

(73) Assignee: ST. JUDE MEDICAL PUERTO RICO LLC, Caguas, PR (US)

(21) Appl. No.: 13/796,785

(22) Filed: Mar. 12, 2013

(51) Int. Cl. A61B 17/00

(52) U.S. Cl. CPC A61B 17/0057
USPC 606/213

(57) ABSTRACT

A tissue puncture closure device having an anchor, a suture, a sealing plug, a compaction assembly, a carrier tube, and an insertion sheath. The suture is connected to the anchor at a distal end of the suture. The sealing plug is slidingly mounted to the suture and positioned proximal of the anchor. The compaction assembly is operable to compact the sealing plug toward the anchor. The carrier tube has the sealing plug positioned therein during delivery to a vessel puncture. The insertion sheath is insertable through the vessel puncture and configured to receive the carrier tube and anchor. At least the carrier tube has a cross-sectional shape with a greater width dimension than a height dimension.

### TECHNICAL FIELD

The present disclosure relates generally to medical devices and more particularly to vascular closure devices.

### BACKGROUND

Various surgical procedures are routinely carried out intravascularly or intraluminally. For example, in the treatment of vascular disease, such as arteriosclerosis, it is a common practice to access the artery and insert an instrument (e.g., a balloon or other type of catheter) to carry out a procedure within the artery. Such procedures usually involve the percutaneous puncture of the artery so that an insertion sheath may be placed in the artery and thereafter instruments (e.g., a catheter) may pass through the sheath and to an operative position within the artery. Intravascular and intraluminal procedures unavoidably present the problem of stopping the bleeding at the percutaneous puncture after the procedure has been completed and after the instruments (and any insertion sheaths used therewith) have been removed. Bleeding from puncture sites, particularly in the case of femoral vascular punctures, may be stopped by utilizing vascular closure devices.

Typical closure devices position an anchor intravascularly spanning the puncture and cover the puncture with a sealing plug to seal the puncture extravascularly. The anchor and sealing plug are typically cinched together with a suture. The anchor is carried to the puncture site within a carrier tube and is thus limited in at least its width dimension to the maximum internal width of the carrier tube. For large bore punctures, the width of the anchor is usually significantly less than the width of the puncture and may not provide adequate wound coverage to create a reliable closure of the puncture. Further, relatively narrow width anchors may have undesirable stress concentrations and be subject to bending at a location where the suture attaches to the anchor. Thus, opportunities exist for improvements in anchor designs for vascular closure devices.

### SUMMARY

One aspect of the present disclosure relates to a tissue puncture closure device having an anchor, a suture, a sealing plug, a compaction assembly, a carrier tube, and an insertion sheath. The suture is connected to the anchor at a distal end of the suture. The sealing plug is slidingly mounted to the suture and positioned proximal of the anchor. The compaction assembly is operable to compact the sealing plug toward the anchor. The sealing plug is positioned in the carrier tube during delivery to a vessel puncture. The insertion sheath is insertable through the vessel puncture and configured to receive the carrier tube and anchor. The carrier tube has a cross-sectional shape with a greater width dimension than a height dimension.

The cross-sectional shape of the insertion sheath may be oval. The carrier tube may have a cross-sectional shape with a greater width dimension than a height dimension. The sealing plug may have a cross-sectional shape with a greater width dimension than a height dimension. The anchor may have a variable width along its length. The anchor may have a variable thickness along its length. The anchor may include a suture attachment point positioned at a midpoint along its length. The anchor may have a maximum width adjacent to the suture attachment point.

Another aspect of the present disclosure relates to a tissue puncture closure device adapted for insertion into and sealing of a tissue puncture. The tissue puncture closure device includes an insertion sheath, a carrier tube, a sealing plug, an anchor having a variable width dimension along its length, and a suture coupling the sealing plug to the anchor and extending through the carrier tube.

The insertion sheath may have a non-circular shaped cross-section. The non-circular shaped cross-section may include an oval shape. The sealing plug may be positioned in the carrier tube, and the anchor and carrier tube may be positioned in the insertion sheath. The insertion sheath and carrier tube may each have a cross-sectional shape with a greater maximum width dimension than a minimum width dimension. The sealing plug may have a non-circular cross-sectional shape. The tissue puncture closure device may include a compaction member configured to compact the sealing plug toward the anchor, wherein the compaction member may have a non-circular cross-sectional shape.

A further aspect of the present disclosure relates to a method of sealing a puncture in a wall of a vessel. The method includes providing a closure device having a sealing plug, an anchor, a suture connected to the anchor, a carrier tube and an insertion sheath, wherein the sealing plug is positioned in the carrier tube and the carrier tube and anchor are positioned in the insertion sheath. At least the carrier tube has a cross-sectional shape with a greater width dimension than height dimension. The method also includes inserting the closure device through the puncture, depositing the anchor within an interior of the vessel, withdrawing the closure device to contact the anchor against an interior surface of the vessel and deposit the sealing plug adjacent to an exterior surface of the vessel, and connecting the sealing plug and anchor together with the suture to seal the puncture.

The method may include arranging the width dimension of the insertion sheath with a maximum width dimension of the puncture prior to inserting the closure device through the puncture. Depositing the anchor may include withdrawing the insertion sheath relative to the carrier tube. The method may include providing at least a portion of the insertion sheath and at least a portion of the carrier tube with the same cross-sectional shape. The method may include providing the anchor with a variable width dimension along its length.

Additional advantages and novel features will be set forth in the description which follows or may be learned by those skilled in the art through reading these materials or practicing the examples disclosed herein.

### BRIEF DESCRIPTION OF THE DRAWINGS

The accompanying drawings illustrate various embodiments of the present disclosure and are a part of the specification. The illustrated embodiments are merely examples and are not intended to be limiting.

FIG. 1 is a side view of a tissue puncture closure device according to the prior art.

FIG. 2 is a side view of the tissue puncture closure device of FIG. 1 inserted into an insertion sheath.

FIG. 2A is a cross-sectional view of a distal end portion of the tissue puncture closure device of FIG. 2 taken along cross-section indicators 2A-2A.

FIG. 3 is a side view of the tissue puncture closure device and insertion sheath of FIG. 2 engaged with a vessel.

FIG. 4 is a side view of the tissue puncture closure device and insertion sheath of FIG. 3 being withdrawn from the vessel to deploy a sealing plug.

FIG. 5 is a side view of the tissue puncture closure device and insertion sheath of FIG. 4 operated manually to compact the sealing plug.

FIG. 6 is a side view of an example tissue puncture closure device inserted into an insertion sheath in accordance with the present disclosure.

FIG. 6A is a cross-sectional view of the tissue puncture closure device of FIG. 6 taken along cross-section indicators 6A-6A.

FIG. 6B is a cross-sectional view of the tissue puncture closure device of FIG. 6 taken along cross-section indicators 6B-6B.

FIG. 7A shows the tissue puncture closure device of FIG. 6 engaged within a vessel.

FIG. 7B is a detailed view of a distal end portion of the tissue puncture closure device of FIG. 7A.

FIG. 8A shows the tissue puncture closure device of FIG. 7A with the sheath retracted to expose the sealing plug.

FIG. 8B is a detailed view of a distal end portion of the tissue puncture closure device of FIG. 8A.

FIG. 9A shows the tissue puncture closure device of FIG. 8A with the compaction member advanced to compact the sealing plug.

FIG. 9B is a detailed view of a distal end portion of the tissue puncture closure device of FIG. 9A.

FIG. 10A shows the tissue puncture closure device of FIG. 9A with the driving assembly released.

FIG. 10B is a detailed view of a distal end portion of the tissue puncture closure device of FIG. 10A.

FIG. 11A is a perspective view of an example anchor for use in the tissue puncture closure device of FIGS. 6-10B.

FIG. 11B is a top view of the anchor of FIG. 11A.

FIG. 11C is a cross-sectional view of the anchor of FIG. 11A taken along cross-section indicators 11C-11C.

FIG. 12A is a perspective view of another example anchor for use in the tissue puncture closure device of FIGS. 6-10B.

FIG. 12B is a top view of the anchor of FIG. 12A.

FIG. 12C is a cross-sectional view of the anchor of FIG. 12A taken along cross-section indicators 12C-12C.

FIGS. 13A-13C show bottom views of example anchors in accordance with the present disclosure positioned relative to a tissue puncture.

FIG. 14 is a perspective view of a distal end portion of the tissue puncture closure device of FIGS. 6-10B aligned with a tissue puncture.

Throughout the drawings, identical reference numbers designate similar, but not necessarily identical, elements.

### DETAILED DESCRIPTION

As mentioned above, vascular procedures are conducted throughout the world and require access to a vessel through a puncture. Most often, the vessel is a femoral artery. To close the puncture following completion of the procedure, many times a closure device is used to sandwich the puncture between an anchor and a sealing plug. However, sometimes the sealing plug is difficult to eject from the closure device and may not properly seat against an exterior situs of the arteriotomy. If the plug and anchor do not seat properly against the arteriotomy, there is a potential for prolonged bleeding. The present disclosure describes methods and apparatus that facilitate positioning of an anchor intravascularly, maximizing coverage of the puncture, and providing oval or elliptical shaped delivery features that accommodate anchors having an increased width.

While the vascular instruments shown and described below include procedural sheaths and puncture sealing devices, the application of principles described herein are not limited to the specific devices shown. The principles described herein may be used with any medical device. Therefore, while the description below is directed primarily to arterial procedures and certain embodiments of a vascular closure device, the methods and apparatus are only limited by the appended claims. Applications of closure devices including those implementing principles described herein include closure of a percutaneous puncture or incision in tissue separating two internal portions of a living body, such as punctures or incisions in blood vessels, ducts or lumens, gall bladders, livers, hearts, etc.

The present disclosure describes a medical device such as a tissue puncture closure device that is capable of retracting a procedural sheath relative to a closure device to expose a distal end of the closure device prior to ejecting a sealing pad. The closure device compacts the sealing pad toward the tissue puncture to seal the tissue puncture. A suture or other filament may be used to cinch the compacted sealing pad and anchor together across a wall of the vessel. The mechanism for compacting the sealing pad may operate automatically upon applying a withdrawal force to the tissue puncture closure device while the anchor abuts against an inner surface of the vessel adjacent to the vessel puncture. The mechanism for compacting the sealing may be selectably engagable and disengageable.

As used in this specification and the appended claims, the term “compact,” “compacting,” or “compaction” is used broadly to mean any type of tamping (i.e., packing down by one or a succession of blows or taps or smooth, steady pressure, but not by excessive force), compacting, or compressing. “Engage” and “engagable” are also used broadly to mean interlock, mesh, or contact between two devices. Likewise “disengage” or “disengageable” means to remove or capable of being removed from interlock, mesh, or contact. A “tube” is an elongated device with a passageway. The passageway may be enclosed or open (e.g., a trough). A “lumen” refers to any open space or cavity in a bodily organ, especially in a blood vessel. The words “including” and “having,” as used in the specification, including the claims, have the same meaning as the word “comprising.”

One aspect of the present disclosure relates to the construction of the anchor member of the closure device. Another aspect of the present disclosure relates to the cross-sectional shape of the carrier tube, bypass tube, locator, and/or delivery sheath used to position the anchor through the vessel puncture. The shape of the carrier tube, bypass tube, locator, and/or delivery sheath may accommodate an increased width for the anchor without increasing the French size of the carrier device. In at least some examples, the cross-sectional shape of the carrier tube, bypass tube, locator, and/or delivery sheath is generally oval or elliptical (referred to generally as a oval throughout). The oval shape of the carrier tube, bypass tube, locator, and/or delivery sheath may permit use of an anchor having an increased width and reduced height (e.g., thickness) as compared to the width and height of the anchor possible when using a circular shaped carrier tube, bypass tube, locator, and/or delivery sheath having the same cross-sectional area.

The increased width of the anchor may provide increased wound coverage across the vessel puncture. The width of the anchor may be increased along its entire length, or selectively widened at certain locations along the length, such as, for example, only at a center portion along the length. A height or thickness of the anchor may be reduced as compared to narrower width designs because the increased width may help distribute the stress over a greater area within the anchor, thus limiting the need for greater thickness. In a design in which the width of the anchor is selectively widened at the center portion only, the increased width may reinforce at least the center of the anchor, which is typically subjected to maximum bending stresses.

The wider portions of the anchor may provide increased wound coverage across a width of the puncture in the artery, which may benefit all size ranges of vessel punctures that are closed by the vascular puncture closure devices having the anchor. Providing a wider anchor may have particular advantages related to closing vessel punctures larger than 10 F, where a larger anchor footprint with a reduced height (e.g., no net cross-sectional area increase compared to existing anchors) is desired.

Changing the cross-sectional shape of the carrier tube, bypass tube, locator, and/or delivery sheath to accommodate an increased width anchor while maintaining the same French size (e.g., cross-sectional area) of the vascular puncture closure device may help accommodate cross-sectional shapes for various features of the vascular puncture closure device that are non-circular. For example, a sealing plug held in the carrier tube and advanced by a compaction member may have a non-circular (e.g., oval or elliptical) cross-sectional shape along at least a portion of its length.

Another aspect of the present disclosure relates to the oval cross-sectional shape of the carrier tube, bypass tube, locator and/or delivery sheath corresponding to the shape of the vascular puncture. An oval-shaped structure typically more closely mirrors the shape of a vascular puncture as compared to a circular-shaped structure. Providing the vascular puncture closure device with an oval cross-sectional shape and orienting a maximum width dimension of the device with a length dimension of the vascular puncture may improve ease of inserting the device into the vascular puncture and reduce damage to the vessel, such as tearing, which otherwise may occur while inserting a circular-shaped device that does not as closely match the shape of the vascular puncture.

Referring now to the drawings, and in particular to FIGS. 1-5, a vascular puncture closure device 100 is shown according to the prior art. The vascular puncture closure device 100 includes a carrier tube 102 with a suture or filament 104 extending at least partially therethrough. The vascular puncture closure device 100 also includes a first or proximal end 106 and a second or distal end 107. External to the distal end 107 of the carrier tube 102 is an anchor 108. The anchor 108 may be an elongated, generally stiff, low profile member including an eye 109 formed at the middle. The anchor 108 is typically made of a biologically resorbable polymer.

The filament 104 is threaded through the anchor 108 and back to a sealing plug 110. The sealing plug 110 may be comprised of, for example, randomly oriented fibrous material bound together by chemical means. The sealing plug 110 is slidingly attached to the filament 104 as the filament passes distally through the carrier tube 102. As the filament traverses the anchor 108 and reenters the carrier tube 102, the filament 104 is securely slip knotted proximal to the sealing plug 110 to facilitate cinching of the sealing plug 110 when the vascular puncture closure device 100 is properly placed and the anchor 108 deployed (see FIG. 4).

The carrier tube 102 typically includes a compaction member 112 disposed therein. The compaction member 112 is slidingly mounted on the filament 104 and may be used by an operator to compact the sealing plug 110 toward the anchor 108 at an appropriate time to seal a vascular puncture 118 within a percutaneous incision 119.

Prior to deployment of the anchor 108 within an artery, the eye 109 of the anchor 108 rests outside the distal end 107 of the carrier tube 102. The anchor 108 may be temporarily held in place flush with the carrier tube 102 by a bypass tube 114 disposed over the distal end 107 of the carrier tube 102 (see FIG. 1). The flush arrangement of the anchor 108 and carrier tube 102 allows the anchor 108 to be inserted into a procedure sheath such as insertion sheath 116 as shown in FIGS. 2-5, and eventually through an vascular puncture 118.

The insertion sheath 116 is shown in FIGS. 2-4 inserted through a percutaneous incision 119 and into a vessel 128. The bypass tube 114 (see FIG. 1) may include an oversized head 120 that prevents the bypass tube 114 from passing through an internal passage of the insertion sheath 116. Therefore, as the vascular puncture closure device 100 is inserted into the insertion sheath 116, the oversized head 120 bears against a proximal surface of a hub portion 117 of insertion sheath 116. Further insertion of the vascular puncture closure device 100 results in sliding movement between the carrier tube 102 and the bypass tube 114, thereby releasing the anchor 108 from the bypass tube 114 (see FIG. 1). Typically, the anchor 108 remains in the flush arrangement shown in FIG. 1 following release from the bypass tube 114, limited in movement by the insertion sheath 116.

The vascular puncture closure device 100 may also include a housing 124 and a pair of sheath connection member 115 that extend distally from the housing 124. The sheath connection members 115 may be constructed to releasably connect the vascular puncture closure device 100 to the insertion sheath 116.

The insertion sheath 116 may include a monofold at a distal end thereof. The monofold acts as a one-way valve to the anchor 108. Typically, monofolds are a plastic deformation in a portion of the insertion sheath 116 that elastically flexes as the anchor 108 is pushed out through the distal end 126 of the insertion sheath 116. Typically, after the anchor 108 passes through the distal end 126 of the insertion sheath 116 and enters the vessel 128, the anchor 108 is no longer constrained to the flush arrangement with respect to the carrier tube 102 and it deploys and rotates to the position shown in FIG. 3.

Referring next to FIGS. 4-5, with the anchor 108 deployed, the vascular puncture closure device 100 and the insertion sheath 116 are withdrawn together, ejecting the sealing plug 110 from the carrier tube 102 into the percutaneous incision 119 and exposing the compaction member 112. Further withdrawal of the vascular puncture closure device 100 fully exposes the compaction member 112 as shown in FIG. 5. The operator can then manually compact the sealing plug 110 while cinching together the anchor 108 and sealing plug 110 with the self-tightening slip-knot on the filament 104. Thus, the tissue puncture is sandwiched between the anchor 108 and the sealing plug 110, thereby sealing the vascular puncture 118. The filament 104 is then cut and the percutaneous incision 119 may be closed. The filament 104, anchor 108, and sealing plug 110 are generally made of resorbable materials and therefore remain in place while the vascular puncture 118 heals.

Using the typical vascular puncture closure device 100 described above, however, it may be difficult to eject and compact the sealing plug 110. The insertion sheath 116 resists deformation as the sealing plug 110 is ejected from the carrier tube and compaction may not commence until the insertion sheath 116 has been removed. Under certain conditions, removal of the insertion sheath 116 prior to compacting the sealing plug 110 may cause the sealing plug 110 to retract or displace proximally from the vascular puncture 118, creating an undesirable gap 121 between the sealing plug 110 and the vascular puncture 118. The gap 121 may remain even after compaction, and sometimes results in only a partial seal and bleeding from the vascular puncture 118.

A portion of the vascular puncture closure device 100 that is inserted into the vascular puncture 118 has a generally circular cross-sectional shape, as shown in FIG. 2A. The insertion sheath 116 has a maximum width W₁ and minimum width W₂ that are substantially the same. A maximum width W₃ of the anchor 108 is limited by the maximum width dimension W₁ of the insertion sheath 116. A thickness T₁ of the anchor 108 may be significant due to the relatively large size of minimum width W₂ of the insertion sheath 116, which is equal to the maximum width W₃.

Referring now to FIGS. 6-10B, an example vascular puncture closure device 200 is shown including a carrier tube 202, a suture 204, an anchor 208, a sealing plug 210 (also referred to as a sealing member or a collagen pad), a compaction member 212, and an insertion sheath 216. The carrier tube 202 is connected to a housing 224. A driving assembly 236 may be held in the housing 224 and operable to advance the compaction member 212 to compact the sealing plug 210. The compaction member 212 and driving assembly 236 may be referred to as a compaction assembly. The carrier tube 202, which carries the sealing plug 210 and anchor 208, is insertable through the insertion sheath 216. The insertion sheath 216 is releasably attached to the housing 224. The housing 224 is positioned at a proximal end 206 of the device, and the anchor 208 and sealing plug 210 are positioned at a distal end 207.

FIGS. 6A and 6B are cross-sectional views taken at distal end locations of the vascular puncture closure device 200. FIG. 6A shows the insertion sheath 216 having a maximum width W₁ and a minimum width dimension W₂. The anchor 208 has a maximum width W₃ and a thickness T₁. Referring to FIG. 6B, the compaction member 212 has a maximum width W₅ and a minimum width W₆. The carrier tube 202 has a maximum width W₇ and a minimum width W₈. Each of the carrier tube 202, compaction member 212, and insertion sheath 216 have a generally oval cross-sectional shape having a maximum width dimension greater than a minimum width dimension. The sealing plug 210 may also have a generally oval shape along at least a portion of its length. FIG. 6A shows the sealing plug 210 having an elongate cross-sectional shape at least along a portion of the carrier tube 202 that has a monofold 203 formed therein to accommodate a portion of the anchor 208 within the distal end portion of the carrier tube 202. In other arrangements, only some of the distal features of the vascular puncture closure device 200 include a non-circular cross-sectional shape. In one example, the insertion sheath 216 has an oval cross-sectional shape and the carrier tube 202 and compaction member 212 have circular cross-sectional shapes.

The cross-sectional area of the vascular puncture closure device 200 at its distal end portion, as shown by the cross sections of FIGS. 6A and 6B, may have the same cross-sectional area as a circular shape. For example, the cross-sectional area of the vascular puncture closure device 100 shown in FIG. 2A has an area:

A=πr²

wherein r=½ of dimensions W₃ and T₁ shown in FIG. 2A.

The cross-sectional area of the elliptical-shaped vascular puncture closure device 200 shown in FIGS. 6A and 6B is:

A=½π(Wₘₐₓ)(Wₘᵢₙ)

wherein the maximum width is W₁ and the minimum width is W₂ shown in FIG. 6A.

Providing the same cross-sectional area for both of the circular and non-circular-shaped vascular puncture closure devices may provide the same French size for the device. The non-circular cross-sectional shape of the vascular puncture closure device 200 shown in FIGS. 6A and 6B provides for an anchor 208 having an increased maximum width W₃ as compared to the maximum width possible for the anchor 108 in a circular cross-sectional shaped vascular puncture closure device.

FIGS. 11A-11C show the anchor 208 having an eye 209, a maximum width W₃, a thickness T₁ and a length L₁. The width W₃ is substantially constant along the length L₁. Similarly, the thickness T₁ may be substantially constant along the length L₁ and across the width W₃. In other arrangements, the thickness T₁ may vary along the length L₁ or across the width W₃. In at least some arrangements, the thickness T₁ may be reduced as compared to the thickness T₁ of the anchor 108 due at least in part to the increased width W₃ of anchor 208 compared to the width W₃ of the anchor 108. The increased width of anchor 208 may provide increased stiffness and reduced stress concentration as compared to a narrower width anchor. The outer profile of the anchor 208 shown in FIG. 11B may be referred to as a capsule shape, an elliptical shape, an oval shape, or more generally as a non-circular shape.

FIGS. 12A-12C show another example anchor 308 having an eye portion 309, a maximum width W₃, a minimum width W₄, a thickness T₁, a total length L₁, a center portion 334, a pair of leg portions 336, and a center portion length L₂. The anchor 308 has an increased width W₃ along only the center portion 334. The legs portions 336 have a minimum width W₄. The minimum width W₄ may be comparable to the maximum width W₃ of the anchor 108. The maximum width W₃ may be comparable to the maximum width of the anchor 208 described above. The length L₂ is typically less than the total length L₁ and may be in the range of, for example, about 25% to about 50% of the total length L₁.

The thickness T₁ is typically constant along the length L₁. In some arrangements, the thickness T₁ may vary along the length L₁, such as, for example, having a greater thickness along the center portion 334 than along the leg portions 336.

FIGS. 13A-13C show the anchors 108, 208, 308 positioned across a vascular puncture 118 having a width W₉. The maximum width W₃ of the anchor 108 leaves gaps X₁ on opposing sides thereof in which the vascular puncture 118 is not covered by the anchor 108. The uncovered portions of the vascular puncture 118 may be more susceptible to leaking (e.g., failure of maintain hemostasis) as part of sealing the vascular puncture 118 with the vascular puncture closure device. FIG. 13B shows the maximum width W₃ of the anchor 208 covering substantially more of the vascular puncture 118 so that the uncovered portions have a reduced gap X₂. FIG. 13C shows the maximum width W₃ of the anchor 308 covering substantially all of the vascular puncture 118 so that the gap X₃ of the uncovered portions is substantially reduced as compared to the arrangement of FIG. 13A.

The increased width W₃ of the anchors 208, 308 not only provides increased coverage of the vascular puncture 118 for purposes of sealing the vascular puncture 118, but also helps more evenly distribute stress within the anchors 208, 308 and reduce stress concentrations in the vessel wall adjacent to the vascular puncture 118.

FIGS. 7A-10B show the vascular puncture closure device 200 in operation sealing a vascular puncture 218 intravascularly. The vascular puncture closure device 200 is first inserted into a percutaneous incision 219 and rotated to align the maximum width dimension W₁ of the insertion sheath 216 with the length dimension L₃ of the vascular puncture 218 as shown in FIG. 14. The vascular puncture closure device 200 is then advanced through the vascular puncture 218 and the insertion sheath 216 is withdrawn to expose the anchor 208 within the vessel interior 230. The vascular puncture closure device 200 is withdrawn in a proximal direction to abut the anchor 208 against an internal wall of the vessel 228 as shown in FIGS. 7A and 7B.

Referring to FIGS. 8A and 8B, a further withdrawal force is applied to the housing 224 of the vascular puncture closure device 200 to withdraw the insertion sheath 216 and carrier tube 202 to expose the sealing plug 210 within the percutaneous incision 219.

A further withdrawal force is then applied to the housing 224 of the vascular puncture closure device 200 as shown in FIG. 9A, which may automatically advance the compaction member 212 to compact the sealing plug 210 toward the anchor 208 to seal vascular puncture 218. The withdrawal of vascular puncture closure device 200, which causes advancing of the compaction member 212, may concurrently cinch a pre-formed knot 205 (see FIG. 10B) formed in suture 204 to hold the sealing plug 210 secured to the anchor 208.

Referring to FIGS. 10A and 10B, the operator may actuate a release member 238, which releases tension in the suture 204 such that the vascular puncture closure device 200 may be withdrawn from the percutaneous incision 219. The suture 204 may then be cut to leave behind the anchor 208 and sealing plug 210 sealing the vascular puncture 218.

Various types of vascular puncture closure devices having different types of driving assemblies (e.g., the driving assembly 236) may benefit from the anchor features disclosed herein and the non-circular cross-sectional shape features of the present disclosure. Some examples of such alternative vascular puncture closure devices are described in U.S. Pat. Nos. 7,618,438; 7,618,436; and 7,749,248, which patents are incorporated herein in their entireties by this reference.

Some potential advantages related to the features disclosed herein include providing increased interarterial coverage by increasing the width of the anchor or selectively increasing the width of a portion of the anchor. The anchor may have a decreased height or thickness to help maintain an overall constant cross-sectional area as compared to narrower width anchors. The present disclosure may maintain or provide improved bending resistance of the anchor when the anchor has at least one of an increased width and a reduced thickness.

Aspects of the present disclosure may be applicable to closing punctures having a size of at least 8 F, but may also be applicable to smaller punctures (e.g., punctures having a size in the range of about 4 F to about 8 F). The oval cross-sectional shape of various features of the vascular puncture closure device disclosed herein may more closely match or mirror the shape of the vascular puncture itself. The increased width and corresponding increased footprint of the anchor that abuts against an internal surface of the vessel may help distribute stress on the vessel wall during pullback of the device (e.g., during compaction of the sealing plug). Various features of the vascular puncture closure device disclosed herein may be applied to extravascular designs and various sealing plug designs.

As discussed above, the increased width of portions of the anchor disclosed herein may provide increased puncture coverage as compared to those anchor designs possible with a vascular puncture closure device having a circular cross-sectional shape at its distal end. The major (e.g., maximum width) axis of the oval cross-sectional shaped features of the vascular puncture closure device may be aligned with a major axis direction (e.g., length dimension) of an vascular puncture to help reduce tearing or other damage to the vessel wall when inserting the vascular puncture closure device. The vascular puncture closure devices disclosed herein may be referred to generally as an oval or elliptical-shaped device and may comprise oval- or elliptical-shaped features such as a sheath, locator, bypass tube, compaction member, and sealing plug.

The preceding description has been presented only to illustrate and describe exemplary embodiments of the present disclosure. It is not intended to be exhaustive or to limit the invention to any precise form disclosed. Many modifications and variations are possible in light of the above teaching. It is intended that the scope of the invention be defined by the following claims.

What is claimed is:

1. A tissue puncture closure device, comprising:
an anchor;
a suture connected to the anchor at a distal end of the suture;
a sealing plug slidingly mounted to the suture and positioned proximal of the anchor;
a compaction assembly operable to compact the sealing plug toward the anchor;
a carrier tube having the sealing plug positioned therein during delivery to a vessel puncture;
an insertion sheath insertable through the vessel puncture and configured to receive the carrier tube and anchor, the insertion sheath having a cross-sectional shape with a greater width dimension than a height dimension.

2. The tissue puncture closure device of claim 1, wherein the cross-sectional shape of the insertion sheath is oval.

3. The tissue puncture closure device of claim 1, wherein the carrier tube has a cross-sectional shape with a greater width dimension than a height dimension.

4. The tissue puncture closure device of claim 1, wherein the sealing plug has a cross-sectional shape with a greater width dimension than a height dimension.

5. The tissue puncture closure device of claim 1, wherein the anchor has a variable width along its length.

6. The tissue puncture closure device of claim 1, wherein the anchor has a variable thickness along its length.

7. The tissue puncture closure device of claim 1, wherein the anchor includes a suture attachment point positioned at a midpoint along its length.

8. The tissue puncture closure device of claim 7, wherein the anchor has a maximum width adjacent to the suture attachment point.

9. A tissue puncture closure device adapted for insertion into and sealing of a tissue puncture, the tissue puncture closure device comprising:
an insertion sheath;
a carrier tube;
a sealing plug;
an anchor having a variable width dimension along its length;
a suture coupling the sealing plug to the anchor and extending through the carrier tube.

10. The tissue puncture closure device of claim 9, wherein the insertion sheath has a non-circular shaped cross-section.

11. The tissue puncture closure device of claim 10, wherein the non-circular shaped cross-section includes an oval shape.

12. The tissue puncture closure device of claim 9, wherein the sealing plug is positioned in the carrier tube, and the anchor and carrier tube are positioned in the insertion sheath.

13. The tissue puncture closure device of claim 9, wherein the insertion sheath and carrier tube each have a cross-sectional shape with a greater maximum width dimension than a minimum width dimension.

14. The tissue puncture closure device of claim 9, wherein the sealing plug has a non-circular cross-sectional shape.

15. The tissue puncture closure device of claim 9, further comprising a compaction member configured to compact the sealing plug toward the anchor, the compaction member having a non-circular cross-sectional shape.

16. A method of sealing a puncture in a wall of a vessel, the method comprising:
providing a closure device having a sealing plug, an anchor, a suture connected to the anchor, a carrier tube and an insertion sheath, the sealing plug being positioned in the carrier tube and the carrier tube and anchor being positioned in the insertion sheath, at least the carrier tube having a cross-sectional shape with a greater width dimension than height dimension;
inserting the closure device through the puncture;
depositing the anchor within an interior of the vessel;
withdrawing the closure device to contact the anchor against an interior surface of the vessel and deposit the sealing plug adjacent to an exterior surface of the vessel;
connecting the sealing plug and anchor together with the suture to seal the puncture.

17. The method of claim 16, further comprising arranging the width dimension of the insertion sheath with a maximum width dimension of the puncture prior to inserting the closure device through the puncture.

18. The method of claim 16, wherein depositing the anchor includes withdrawing the insertion sheath relative to the carrier tube.

19. The method of claim 16, further comprising providing at least a portion of the insertion sheath and at least a portion of the carrier tube with the same cross-sectional shape.

20. The method of claim 16, further comprising providing the anchor with a variable width dimension along its length.
